(19) United States
(12) Patent Application Publication
(10) Pub. No.: US 2026/0076887 A1
(43) Pub. Date: Mar. 19, 2026

(54) PERSONAL CARE COMPOSITIONS CONTAINING TAILORED MONOLIPID-RHAMNOLIPIDS AND SULFATE-FREE SURFACTANTS

(71) Applicant: THE PROCTER & GAMBLE COMPANY, Cincinnati, OH (US)

(72) Inventors: Howard David HUTTON, III, Oregonia, OH (US); Isoken Omosefe IGWEKALA-NWEKE, Springdale, OH (US); John Samuel KENNEY, Dayton, OH (US); Debora W. Chang, Mason, OH (US); Sumanth Narahari Jamadagni, West Chester, OH (US)

(21) Appl. No.: 19/397,151

(22) Filed: Nov. 21, 2025

Related U.S. Application Data
(63) Continuation-in-part of application No. 19/062,173, filed on Feb. 25, 2025.
(60) Provisional application No. 63/557,812, filed on Feb. 26, 2024.
(60) Provisional application No. 63/558,670, filed on Feb. 28, 2024.
(60) Provisional application No. 63/712,718, filed on Oct. 28, 2024.
(60) Provisional application No. 63/719,856, filed on Nov. 13, 2024.
(60) Provisional application No. 63/719,861, filed on Nov. 13, 2024.

(51) Int. Cl. A61K 8/60; A61K 8/365; A61K 8/44; A61K 8/46; A61Q 5/02

(52) U.S. Cl. CPC A61K 8/604; A61K 8/365; A61K 8/442; A61K 8/466; A61Q 5/02; A61K 2800/596; A61K 2800/75

(57) ABSTRACT

A personal care composition, comprising a mono-rhamno-mono-lipid, a di-rhamno-mono-lipid, a co-surfactant and a carrier. The combination of rhamno-mono-lipids and co-surfactants in a personal cleansing composition reduces the eye irritation potential during application.

### CROSS REFERENCE TO RELATED APPLICATIONS

This application is a continuation-in-part of U.S. patent application Ser. No. 19/062,173 filed Feb. 25, 2025, which claims the benefit, under 35 U.S.C. § 119(e), of U.S. Provisional Patent Application Ser. Nos. 63/557,812 filed Feb. 26, 2024, 63/558,670 filed Feb. 28, 2024, 63/712,718 filed Oct. 28, 2024, and 63/719,861 filed Nov. 13, 2024, and 63/719,856 filed Nov. 13, 2024, the entire disclosure of all which is fully incorporated by reference herein.

### FIELD

The present disclosure generally relates to a personal care composition containing a tailored rhamnolipid surfactant in combination with a co-surfactant. More specifically, the present disclosure relates to personal care composition comprising a rhamno-mono-lipids and co-surfactants that provide good lather properties.

### BACKGROUND

Personal cleansing compositions, such as shampoos and body washes, are commonly used to restore cleanliness and enhance the appearance of hair and skin. These compositions are necessary due to the accumulation of dirt, sebum, and other environmental contaminants that can negatively impact the look and feel of hair and skin. During typical use, these compositions may run off into the eye area, which may cause discomfort and/or irritation. In recent years, there has been a growing demand for personal care products that incorporate milder cleansers (that reduce eye irritation), environmentally friendly ingredients, and fewer overall ingredients.

One area of interest is the use of non-sulfated surfactants. While these surfactants are preferred by some consumers to avoid the perceived harshness of sulfated surfactants, they often exhibit drawbacks in terms of their ability to form coacervates with conditioning polymers, resulting in suboptimal lathering, cleansing, and conditioning performance. Additionally, certain cationic conditioning polymers can introduce instability into sulfate-free surfactant systems, leading to the formation of undesired in situ coacervates that negatively impact product appearance and performance.

Glycolipid surfactants, such as rhamnolipids and sophorolipids, have recently gained attention for their perceived environmental friendliness. However, they have seen limited use in personal care compositions due, at least in part, to concerns about inferior foaming and cleaning performance.

Thus, there is a need for improved personal cleansing compositions that strike a balance between mildness, environmental friendliness, effective cleaning and foaming properties. Accordingly, it would be desirable to provide a personal cleansing composition that includes a rhamnolipid surfactant that provides good foaming and cleaning properties for reducing eye irritation.

### SUMMARY

Disclosed herein is a personal care composition, comprising:

A personal care composition comprising:
a) a mono-rhamno-mono-lipid of structure I:

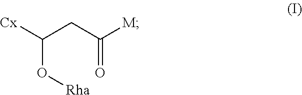

b) a di-rhamno-mono-lipid of structure II:

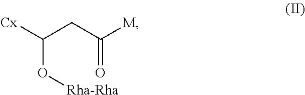

wherein:
Rha is rhamnose,
Cx is a C4-C22 alkyl, aryl, heteroalkyl, heteroaryl, unsaturated alkenyl, or unsaturated heteroalkenyl, and
M is a OH, alkyl, heteroalkyl, aryl, heteroaryl, hetero arylalkyl, arylalkyl, tauryl, O—X+, wherein X+ is a cation, or O—R1, wherein R1 is selected from an alkyl, branched alkyl, and cyclic alkyl, and stereoisomers thereof;
wherein the personal care composition comprises a Log EC50 value of greater than about 0.3.

### BRIEF DESCRIPTION OF THE DRAWINGS

While the specification concludes with claims particularly pointing out and distinctly claiming the subject matter of the present invention, it is believed that the invention can be more readily understood from the following description taken in connection with the accompanying drawings, in which:

FIG. 1 represents schematically the reduced ocular irritation/cytotoxicity to corneal epithelial cells of Inventive Examples E1-E3 compared to Comparative Examples C1-C4.

### DETAILED DESCRIPTION OF THE INVENTION

Certain rhamnolipids are known to have antimicrobial activity and generate good lather during use. However, commercially available rhamnolipids for use in mass produced personal care compositions such as shampoos, conditioners and body washes are primarily rhamno-di-lipids, which is to say they have two lipid tails because it generally believed that rhamno-di-lipids are better surfactants than rhamno-mono-lipids. It has now been surprisingly discovered that certain rhamno-mono-lipids provide better efficacy than rhamno-di-lipids in personal cleansing compositions without undesirably impacting the lather properties of the composition. It has also been surprisingly discovered that these rhamno-mono-lipids provide reduced ocular (eye) irritation as compared to rhamno-di-lipids and sulfated surfactants. Further, it has been surprisingly found that such rhamno-mono-lipids synergistically combined with particular co-surfactant(s) provided improved lather properties and ocular irritation reduction over rhamno-di-lipids when combined with the same particular co-surfactant(s). Improved lather enhances distribution, cleaning efficiency, and rinsability, while reduced ocular irritation minimizes stinging and tearing to improve safety, comfort, and user compliance. Collectively improving lather while reducing ocular irritation represents a meaningful performance and tolerability advancement over compositions that typically trade off foam quality against eye sensitivity.

Reference within the specification to “embodiment(s)” or the like means that a particular material, feature, structure and/or characteristic described in connection with the embodiment is included in at least one embodiment, optionally a number of embodiments, but it does not mean that all embodiments incorporate the material, feature, structure, and/or characteristic described. Furthermore, materials, features, structures and/or characteristics may be combined in any suitable manner across different embodiments, and materials, features, structures and/or characteristics may be omitted or substituted from what is described. Thus, embodiments and aspects described herein may comprise or be combinable with elements or components of other embodiments and/or aspects despite not being expressly exemplified in combination, unless otherwise stated or an incompatibility is stated.

All ingredient percentages described herein are by weight of the cosmetic composition, unless specifically stated otherwise, and may be designated as “wt %.” All ratios are weight ratios, unless specifically stated otherwise. All ranges are inclusive and combinable. The number of significant digits conveys neither a limitation on the indicated amounts nor on the accuracy of the measurements. All numerical amounts are understood to be modified by the word “about” unless otherwise specifically indicated. Unless otherwise indicated, all measurements are understood to be made at approximately 25° C. and at ambient conditions, where “ambient conditions” means conditions under about 1 atmosphere of pressure and at about 50% relative humidity. All numeric ranges are inclusive of narrower ranges, and delineated upper and lower range limits are interchangeable to create further ranges not explicitly delineated.

The compositions of the present invention can comprise, consist essentially of, or consist of, the essential components as well as optional ingredients described herein. As used herein, “consisting essentially of” means that the composition or component may include additional ingredients, but only if the additional ingredients do not materially alter the basic and novel characteristics of the claimed compositions or methods. As used in the description and the appended claims, the singular forms “a,” “an,” and “the” are intended to include the plural forms as well, unless the context clearly indicates otherwise.

### Definitions

“About” modifies a particular value by referring to a range of plus or minus 20% or less of the stated value (e.g., plus or minus 15% or less, 10% or less, or even 5% or less).

“Antimicrobial” means a material that prevents or inhibits the growth of and/or kills microorganisms (e.g., bacteria or fungi).

“Apply” or “application,” as used in reference to a composition, means to apply or spread the composition onto a human keratinous surface such as the skin or hair.

“Cleansing composition” refers to a personal care composition intended for use in cleaning a bodily surface. Some non-limiting examples of cleansing compositions are shampoos, conditioners, conditioning shampoos, shower gels, liquid hand cleansers, facial cleansers, and the like.

“Personal care composition” is meant a product, which in the ordinary course of usage is applied to or contacted with a body surface to provide a beneficial effect such as, for example, improving appearance, cleansing, and odor control. Body surface includes skin, hair, teeth, or nails. Some non-limiting examples of personal care compositions include oral care compositions, (e.g., dentifrice, mouth rinse, mouth spray, lozenge, chewable tablet, chewing gum, teeth whitening strips, floss and floss coatings, breath freshening dissolvable strips, denture care products, and denture adhesive products), shave care compositions (e.g., after shave gels and creams, pre-shave preparations, shaving gels, creams), cough and cold compositions, leave-on skin lotions and creams, shampoos, body washes, hair conditioners, hair dyeing and bleaching compositions, styling mousses, shower gels, bar soaps, hand soaps, antiperspirants, deodorants, depilatories, lipsticks, foundations, mascara, sunless tanners and sunscreen lotions, feminine care compositions and absorbent articles, baby care compositions and absorbent articles.

“Substantially free of” means a composition or ingredient comprises less than 3% of a subject material, by weight of the composition or ingredient (e.g., less than 2%, less than 1% or even less than 0.5%). “Free of” means a composition or ingredient contains 0% of a subject material.

“Synergy” and variations thereof mean that the effect provided by a combination of two or more materials (e.g., a combination of rhamno-di-lipid and rhamno-mono-lipid and/or a combination of rhamno-mono-lipids with different carbon chain lengths) is more than the additive effect expected for these materials.

### Personal Care Composition

The personal care compositions herein include a rhamnolipid and co-surfactant that provides consumer acceptable lather/cleansing, conditioning and/or antimicrobial properties.

### Rhamnolipids

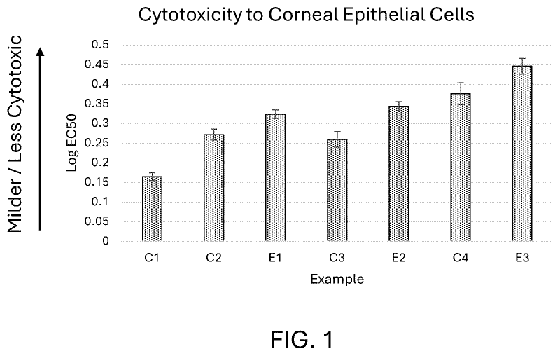

Rhamnolipids include a glycosyl head group (rhamnose) and a fatty acid tail (lipid). The resulting molecular structure imparts amphiphilic properties essential for their effectiveness as surfactants. The two main classes of rhamnolipids are mono-rhamnolipids and di-rhamnolipids, which consist of one or two rhamnose head groups, respectively. Each class of rhamnolipid can have either one or two lipid tails (mono-lipids or di-lipids, respectively), as exemplified in FIG. 1. A more general description of glycolipid surfactants can be found in U.S. Publication No. 2023/0320961.

The personal care compositions herein include a mixture of rhamno-mono-lipids for delivering a lathering/cleansing benefit, reduction in ocular irritation benefit, and an antimicrobial benefit. In some instances, the rhamnolipid surfactant system may also deliver a conditioning benefit. Multifunctional ingredients like the rhamnolipids herein can be used to formulate personal care compositions that have fewer ingredients, which is desired by some consumers.

The rhamno-mono-lipid mixture can be made using a semi-synthetic method that involves performing a base hydrolysis on a rhamno-di-lipid compound (e.g., Rheance® One brand rhamnolipids from Evonik). Possible bases include sodium hydroxide (NaOH), potassium hydroxide (KOH), calcium hydroxide (CaOH), cesium hydroxide (CsOH), magnesium hydroxide (MgOH, ammonium hydroxide (NH4OH), and alkylamine containing bases such as triethylamine (Et3N). A semi-synthetic approach for hydrolyzing rhamno-di-lipids is exemplified in FIG. 2.

The rhamno-mono-lipids resulting from the hydrolysis of a rhamno-di-lipid species may include 25% or more, based the total weight of the rhamnolipids, of rhamno-mono-lipids (e.g., greater than or equal to about 30%, about 35%, about 40%, about 45%, about 50%, about 55%, about 60%, about 65%, about 70%, about 75%, about 80%, about 85%, about 90%, about 95%, about 96%, about 97%, about 98%, about 99%, or even about 100%). These exemplary amounts of rhamno-mono-lipid species may be any combination of di-rhamno-mono-lipid and mono-rhamno-mono-lipid species, including only a single species of rhamno-mono-lipid. In some instances, the weight ratio of mono-rhamno-mono-lipid to di-rhamno-mono-lipid in the composition may range from about 1:10 to about 10:1 (e.g., about 1:5 to about 5:1, about 1:4 to about 4:1, about 1:3 to about 3:1, about 1:2 to about 2:1 or even about 1:1).

The reaction product of di-rhamno-di-lipid hydrolysis may be free of or substantially free of di-lipid species (i.e., di-rhamno-di-lipid and mono-rhamno-di-lipid species). In other words, the hydrolysis reaction may completely convert the di-lipid species to mono-lipid species. In some instances, the di-lipid species may not be completely hydrolyzed, and it may be desirable to remove the di-lipid species using conventional methods known in the art. In some instances, the preservative system herein may include less than about 25% of a di-lipid species, based on the total weight of the rhamnolipids (e.g., less than about 20%, about 15%, about 10%, about 5%, about 4%, about 3%, about 2%, about 1% or even 0%).

Some non-limiting examples of methods for making the rhamno-mono-lipids herein, including suitable methods for hydrolyzing rhamno-di-lipids, are disclosed in U.S. Provisional Ser. Nos. 63/558,670, 63/557,812 and 63/641,630.

The reaction product resulting from the hydrolysis of the di-rhamno-di-lipid species includes compounds (c), (d) and (e), and all stereo isomers thereof, as shown below:

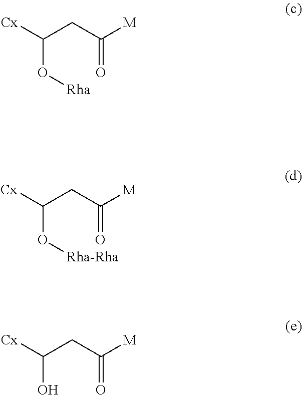

wherein Rha is rhamnose;
wherein each Cx is independently selected from an alkyl, aryl, heteroalkyl, heteroaryl, unsaturated alkenyl, and unsaturated heteroalkenyl;
wherein each Cx independently has a carbon chain length from 4 to 22 (e.g., 5 to 13, 10 or 12);
wherein each M is independently selected from OH; O—X+ wherein X+ is a cation; OR1;
alkyl, heteroalkyl; aryl; heteroaryl; hetero arylalkyl; arylalkyl; and tauryl;
wherein R1 is selected from an alkyl, branched alkyl, and cyclic alkyl.

Some nonlimiting examples of mono-rhamno mono-lipid that may be suitable for use herein include 3-(((2R,3R,4R,5R,6S)-3,4,5-trihydroxy-6-methyltetrahydro-2H-pyran-2-yl)oxy)hexadecanoic acid, 3-(((2R,3R,4R,5R,6S)-3,4,5-trihydroxy-6-methyltetrahydro-2H-pyran-2-yl)oxy)octenoic acid, 3-(((2R,3R,4R,5R,6S)-3,4,5-trihydroxy-6-methyltetrahydro-2H-pyran-2-yl)oxy)decenoic acid, 3-(((2R,3R,4R,5R,6S)-3,4,5-trihydroxy-6-methyltetrahydro-2H-pyran-2-yl)oxy)octanoic acid, 3-(((2R,3R,4R,5R,6S)-3,4,5-trihydroxy-6-methyltetrahydro-2H-pyran-2-yl)oxy)tetradecanoic acid, 3-(((2R,3R,4R,5R,6S)-3,4,5-trihydroxy-6-methyltetrahydro-2H-pyran-2-yl)oxy)decanoic acid, 3-(((2R,3R,4R,5R,6S)-3,4,5-trihydroxy-6-methyltetrahydro-2H-pyran-2-yl)oxy)dodecanoic acid, 3-(((2R,3R,4R,5R,6S)-3,4,5-trihydroxy-6-methyltetrahydro-2H-pyran-2-yl)oxy)dodecen-dienoic acid, and stereoisomers thereof.

Some non-limiting examples of di-rhamno mono-lipid that may be suitable for use herein include 3-(((2R,3R,4R,5R,6S)-4,5-dihydroxy-6-methyl-3-(((2R,3S,4S,5S,6S)-3,4,5-trihydroxy-6-methyltetrahydro-2H-pyran-2-yl)oxy)tetrahydro-2H-pyran-2-yl)oxy)hexadecanoic acid, 3-(((2R,3R,4R,5R,6S)-4,5-dihydroxy-6-methyl-3-(((2R,3S,4S,5S,6S)-3,4,5-trihydroxy-6-methyltetrahydro-2H-pyran-2-yl)oxy)tetrahydro-2H-pyran-2-yl)oxy)tetradecanoic acid, 3-(((2R,3R,4R,5R,6S)-4,5-dihydroxy-6-methyl-3-(((2R,3S,4S,5S,6S)-3,4,5-trihydroxy-6-methyltetrahydro-2H-pyran-2-yl)oxy)tetrahydro-2H-pyran-2-yl)oxy)decenoic acid, 3-(((2R,3R,4R,5R,6S)-4,5-dihydroxy-6-methyl-3-(((2R,3S,4S,5S,6S)-3,4,5-trihydroxy-6-methyltetrahydro-2H-pyran-2-yl)oxy)tetrahydro-2H-pyran-2-yl)oxy)tetradecenoic acid, 3-(((2R,3R,4R,5R,6S)-4,5-dihydroxy-6-methyl-3-(((2R,3S,4S,5S,6S)-3,4,5-trihydroxy-6-methyltetrahydro-2H-pyran-2-yl)oxy)tetrahydro-2H-pyran-2-yl)oxy)decanoic acid, 3-(((2R,3R,4R,5R,6S)-4,5-dihydroxy-6-methyl-3-(((2R,3S,4S,5S,6S)-3,4,5-trihydroxy-6-methyltetrahydro-2H-pyran-2-yl)oxy)tetrahydro-2H-pyran-2-yl)oxy)octanoic acid, and 3-(((2R,3R,4R,5R,6S)-4,5-dihydroxy-6-methyl-3-(((2R,3S,4S,5S,6S)-3,4,5-trihydroxy-6-methyltetrahydro-2H-pyran-2-yl)oxy)tetrahydro-2H-pyran-2-yl)oxy)dodecanoic acid, and stereoisomers thereof.

The personal care compositions herein may be free of or substantially free of rhamno-di-lipids. However, in some instances it may be desirable to include a rhamno-di-lipid (mono-rhamno and/or di-rhamno) because it has been surprisingly discovered that the amount of rhamno-di-lipid and/or ratio of rhamno-mono-lipid to rhamno-di-lipid can be tailored to achieve synergistic lather benefits, as discussed in more detail in the examples below, without sacrificing anti-microbial efficacy. In some instances, the composition may include a ratio of rhamno-di-lipid to a total amount of rhamno-mono-lipid of less than about 3:1 (e.g., about 4:1, about 5:1, about 6:1, about 7:1, about 8:1, about 9:1 or even about 10:1 or less).

The ratio of the combined total amount of a mono-rhamno-mono-lipid of structure (c) and a di-rhamno-mono-lipid of structure (d) to the total amount of hydroxypropyl methylcellulose thickener of structure A is about 6:5 to about 12:1.5.

The ratio of the combined total amount of a mono-rhamno-mono-lipid of structure (c) and a di-rhamno-mono-lipid of structure (d) to the total amount of an acrylic polymer thickener of Structure B is about 6:5 to about 12:3.5.

The personal cleansing composition may have a viscosity in the range of about 2500 centipoise (cp) to about 14,000 cp, or about 3,000 cp to about 12,000 cp.

The rhamnolipid may be present in the composition at about 0.5 wt. % to about 30 wt. % (e.g., about 1 wt. % to about 25 wt. %, about 2 wt. % to about 20 wt. %, about 3 wt. % to about 15 wt. %, or about 5 wt. % to about 10 wt. %), based on the weight of the personal care composition. The personal care composition may, optionally, include an additional surfactant (e.g., anionic, non-ionic and/or amphoteric surfactant) and/or other ingredients commonly found in compositions of the type described.

### Co-Surfactant

The personal care composition herein includes a co-surfactant selected from anionic surfactants, amphoteric surfactants, zwitterionic surfactants, non-ionic surfactants and combinations of these. As discussed above, it has been found that combining co-surfactants with rhamno-mono-lipids provide improved lather properties and improved ocular irritation reduction as compared to combining the same co-surfactants with rhamno-di-lipids.

Some non-limiting examples of anionic surfactants include non-sulfated anionic surfactants such as isethionates, carboxylates, sulfonates (e.g., alpha olefin sulfonates, linear alkylbenzene sulfonates, alkyl glyceryl sulfonates, sodium laurylglucosides hydroxypropylsulfonate), sulfosuccinates, sulfoacetates, sulfolaurates, amino acid-based surfactants (e.g., glycinates, taurates, alaninates, glutamates), lactate- and lactylate-based surfactants (e.g., sodium lauroyl lactate and sodium lauroyl lactalyte), phosphate ester surfactants and combinations thereof.

The co-surfactant may include an acyl taurate surfactant. The acyl taurate surfactants herein may be saturated or unsaturated. Acyl taurate surfactants that may be suitable for use herein are generally described by Formula F below.

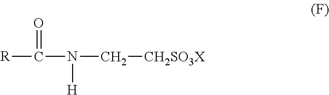

Where: R may be an alkyl group with 5 to 23 carbon atoms (e.g., 7-21, 7-17, 7-15, 7-13, 11-17, 11-15, 11-13 or even 11 carbon atoms) and X may be a suitable counterion (e.g., sodium, potassium, magnesium, ammonium or triethanolamine).

Some nonlimiting examples of acyl taurates are capric ester taurate, cocoyl taurate, lauroyl taurate, myristoyl taurate, caproyl taurate, oleoyl taurate, capryloyl taurate, palmitoyl taurate, stearoyl taurate, linoleoyl taurate, salts of these and combinations thereof. An example of a suitable acyl taurate salt is sodium lauroyl taurate.

The acyl taurate surfactant may be present in the anionic surfactant at an amount of 60% to 90% (e.g., 65% to 85%, 70% to 80%, or even about 75%) by weight, based on the total weight of the anionic surfactant.

The anionic surfactant may include an N-alkyl acyl taurate surfactant. The N-alkyl acyl taurate surfactants herein may be either saturated or unsaturated. The N-alkyl acyl taurate surfactants herein are generally described by Formula G below.

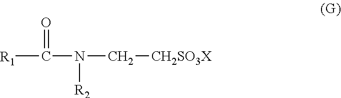

Where: R1 may be an alkyl group with 5 to 23 carbon atoms (e.g., 7-21, 7-17, 7-15, 7-13, 11-17, 11-15, 11-13 or even 11 carbon atoms) and X may be a suitable counterion (e.g., sodium, potassium, magnesium, ammonium or triethanolamine) and R2 may be an alkyl group with 1 to 4 carbon atoms. Some nonlimiting examples of N-alkyl acyl taurates that may be suitable for use herein include methyl capric ester taurate, methyl cocoyl taurate, methyl lauroyl taurate, methyl myristoyl taurate, methyl caproyl taurate, methyl oleoyl taurate, methyl capryloyl taurate, methyl palmitoyl taurate, methyl stearoyl taurate, methyl linoleoyl taurate and combinations thereof. An example of a suitable N-alkyl acyl taurate salt is sodium methyl lauroyl taurate. The N-alkyl acyl taurate surfactant may be present in the at an amount of 1% to 10% (e.g., 2% to 9%, 3% to 8%, 4% to 7% or 5% to 6%) by weight, based on the weight of the anionic surfactant.

Acyl taurate and N-alkyl acyl taurate surfactants used in the personal care compositions herein may be selected from the above examples and combinations thereof.

The anionic surfactant may include an alpha olefin sulfonate surfactant. The alpha olefin sulfonate surfactants herein may be saturated or unsaturated and are generally described by Formula H below.

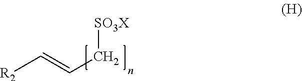

Where: R3 may be an alkyl group with 5 to 23 carbon atoms (e.g., 7 to 21, 7 to 17, 7 to 15, 7 to 13, 11 to 17, 11 to 15, 11 to 13, 14 to 16, or even 11 carbon atoms) and X may be a suitable counterion (e.g., sodium, potassium, magnesium, ammonium or triethanolamine) and n may be 1 or 2. According to various aspects of the disclosure, the alpha olefin sulfonate surfactant may be a C14 to C16 alpha olefin sulfonate surfactant, in which case R3 may be comprise from 10 to 13 carbon atoms.

The alpha olefin sulfonate surfactant may be present in the anionic surfactant at an amount of about 50 wt. % to about 75 wt. % based on the weight of the anionic surfactant.

The anionic surfactant may comprise one or more amino acid-based surfactants, such as glycinates, alaninates, and glutamates. Amino-acid based surfactants may offer good foaming properties, contributing to a pleasant cleansing experience, and leave the skin and hair feeling soft and conditioned. The amino acid-based surfactant may be present at an amount of about 50% to about 75% by weight, based on the weight of the anionic surfactant.

Non-limiting examples of glutamates include lauroyl glutamate, cocoyl glutamate, myristoyl glutamate, palmitoyl glutamate, stearoyl glutamate. Examples of glutamate salts include sodium cocoyl glutamate, disodium cocoyl glutamate, sodium lauroyl glutamate, potassium cocoyl glutamate, triethanolamine cocoyl glutamate, sodium myristoyl glutamate, sodium palmitoyl glutamate, sodium stearoyl glutamate, ethoxylated alkyl glutamates, and combinations thereof.

Non-limiting examples of alaninates include lauroyl alaninate, cocoyl alaninate, myristoyl alaninate, palmitoyl alaninate, stearoyl alaninate, and combinations thereof. Examples of some specific alaninate salts include sodium cocoyl alaninate, sodium lauroyl alaninate, potassium cocoyl alaninate, triethanolamine cocoyl alaninate, sodium myristoyl alaninate, sodium palmitoyl alaninate, sodium stearoyl alaninate.

Non-limiting Examples of glycinates include lauroyl glycinate, cocoyl glycinate, myristoyl glycinate, palmitoyl glycinate, stearoyl glycinate, and combinations thereof. Examples of amino-acid based surfactant salts include sodium cocoyl glycinate, potassium cocoyl glycinate, and sodium lauroyl glycinate.

Amino-acid based surfactants, when included in the personal care composition, may be selected from the above examples or combinations thereof.

Anionic co-surfactants that may be suitable for use herein include non-taurate, non-sulfate anionic surfactants such as carboxylates, sulfonates (e.g., linear alkylbenzene sulfonates, alkyl glyceryl sulfonates, sodium laurylglucosides hydroxypropylsulfonate), branched alkyl sulfates, sulfosuccinates (e.g., ethoxylated alkyl sulfosuccinates), sulfoacetates, sulfolaurates, lactate- and lactylate-based surfactants (e.g., sodium lauroyl lactate and sodium lauroyl lactalyte), phosphate ester surfactants, and combinations of these.

Zwitterionic surfactants are surfactants whose polar functional group has two permanent charges that do not change with changing pH. Amphoteric surfactants have polar functional groups whose charge depends on the pH of the solution and can exhibit different charges as the pH changes from acid to neutral to basic, ranging from cationic to zwitterionic and potentially even to anionic. It is to be appreciated that the terms “zwitterionic” and “amphoteric” may apply to a single compound. For example, the terms both apply to CAPB, because CAPB's amphoteric behavior stems from its zwitterionic structure. The presence of both positive and negative charge centers in CAPB allows it to react as both an acid (by donating a proton in basic conditions) and a base (by accepting a proton in acidic conditions).

In some aspect, the amphoteric and/or zwitterionic surfactant may be a derivative of an aliphatic secondary and tertiary amines in which one of the aliphatic substituents contains from 8 to 18 carbon atoms and one aliphatic substituent contains an anionic group such as a carboxy, sulfonate, phosphate, or phosphonate group. Non-limiting examples of zwitterionic surfactants includes sultaines and betaines. Non-limiting examples of amphoteric surfactants include amphoacetates, amphodiacetates and propionates.

It may be desirable for the co-surfactant to include a betaine. Some non-limiting examples of betaines that may be suitable include alkyl betaines, alkylamido betaines, amine oxide betaines, sulfobetaines and combinations thereof. Some non-limiting examples of alkyl betaines include cetyl betaine, behenyl betaine, erucyl betaine, dodecyl betaine, tetradecyl betaine, hexadecyl betaine, and octadecyl betaine. Examples of alkylamido betaines include but are not limited to lauramidopropyl betaine (LAPB), myristamidopropyl betaine, palmitamidopropyl betaine, stearamidopropyl betaine, oleamidopropyl betaine, ricinoleamidopropyl betaine, cocoamidopropyl betaine (CAPB), and coco betaine. Examples of amine oxide betaines include but are not limited to lauramine oxide, myristamine oxide, palmitamine oxide, stearamine oxide, and cocoamidoamine oxide. CAPB and LAPB may be particularly suitable co-surfactants for use herein.

Some non-limiting examples of amphoteric and/or zwitterionic surfactants include derivatives of aliphatic secondary and tertiary amines in which one of the aliphatic substituents contains from 8 to 18 carbon atoms and one aliphatic substituent contains an anionic group such as a carboxy, sulfonate, phosphate, or phosphonate group. For example, amphoacetates (e.g., cocoamphoacetate, lauroamphoacetate), amphodiacetates (e.g., cocoamphodiacetate, lauroamphodiacetate), betaines, amidobetaines (e.g., cocamidopropyl betaine and lauramidopropyl betaine), amidosulfobetaines, propionates, sultaines, hydroxysultaines (e.g., amidopropyl hydroxysultaines), and combinations thereof. An exemplary amphoacetates may include, for example, sodium lauroamphoacetate (NaLAA). The co-surfactant may include a non-ionic surfactant, selected from, for example, glyceryl esters of alkanoic acids, polyglyceryl esters of alkanoic acids, propylene glycol esters of alkanoic acids, sorbitol esters of alkanoic acids, alkanolamides, alkoxylated amides, alkyl glycosides, alkyl glucosides, alkyl polyglucosides acyl glucamides, amine oxides and combinations thereof. Some particularly suitable examples of non-ionic surfactants include cocamide, cocamide monoethanolamine (MEA), PPG-2 cocamide, PPG-2 hydroxyethyl cocamide, PPG-2 hydroxyethyl isostearamide, lauroyl/myristoyl methyl glucamide, capryloyl/caproyl methyl glucamide, cocoyl methyl glucamide, decyl glucoside, coco-glucoside, lauryl glucoside, lauramine oxide, cocamine oxide and combinations thereof. Cocamide MEA may be particularly suitable for use as an non-ionic surfactant, for example.

More specific examples of the optional co-surfactants described above are disclosed in U.S. Publication No. 2019/0105246, U.S. Publication No 2018/0098923, U.S. Pat. No. 9,271,908, PCT Publication No. WO 2020/016097, and McCutcheon's Emulsifiers and Detergents, 2019, MC Publishing Co.

The co-surfactants, when present, may be included in the personal care compositions to provide the desired cleaning and lather performance. Any additional surfactants should be physically and chemically compatible with the other components of the personal care compositions described herein and should not otherwise unduly impair product stability, aesthetics, or performance.

Co-surfactants may be present in the personal care compositions at about 0.5 wt. % to about 30 wt. % (e.g., about 1 wt. % to about 25 wt. %, about 2 wt. % to about 20 wt. %, about 3 wt. % to about 15 wt. %, or about 5 wt. % to about 10 wt. %), based on the weight of the personal care composition.

The total amount of all surfactant, including the rhamnolipid and co-surfactant, in the personal care composition may be in the range of about 5 wt. % to about 50 wt. %, or about 6 wt. % to about 40 wt. %, or about 7 wt. % to about 30 wt. %, or about 10 wt. % to about 25 wt. %. The ratio of total rhamnolipids to total co-surfactants may be in the range of about 10:1 to about 1:10, or about 5:1 to about 1:5, or about 2:1 to about 1:2.

The personal care composition may contain less than about 10% of sulfated surfactants, if present at all, such as sodium lauryl sulfate and sodium laureth sulfate (e.g., less than about 8%, about 7%, about 5%, about 3%, about 2%, about 1% or even 0%).

### Associative Thickener

As such, the personal cleansing composition of the present invention includes an associative thickener. One associative thickener that has been found to provide a synergistic thickening effect when combined with a rhamno-mono-lipid surfactant system includes a hydroxypropyl methylcellulose associative thickener of Structure A.

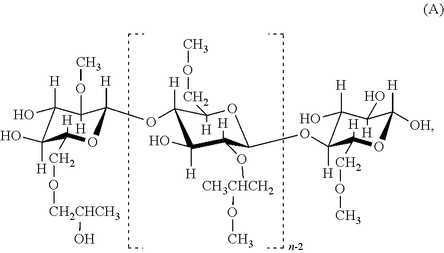

wherein structure (A) comprises:
less than about 25 wt. % methoxyl groups;
greater than about 15 wt. % hydroxypropoxyl groups.

When Structure A is used as the associative thickener of the present invention, the associative thickener may be present in the personal cleansing composition at a level of about 1.5 wt. % to about 5 wt. %, or about 1.5 wt. % to about 4 wt. %, or about 1.5 wt. % to about 3 wt. %, or about 2 wt. % to about 3 wt. %.

A second associative thickener that has been found to provide a synergistic thickening effect when combined with a rhamno-mono-lipid surfactant system includes an acrylic polymer associative thickener having the following Structure B:

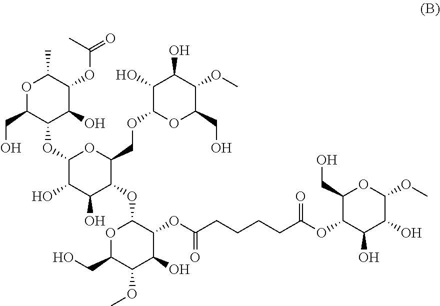 [SMILES: CO[C@H]1O[C@H](CO)[C@@H](OC(=O)CCCCC(=O)O[C@H]2[C@@H](O[C@@H]3[C@@H](O)[C@H](O)[C@H](O[C@H]4[C@H](O)[C@@H](OC(C)=O)[C@@H](C)O[C@@H]4CO)O[C@H]3CO[C@H]3O[C@H](CO)[C@@H](OC)[C@H](O)[C@H]3O)O[C@H](CO)[C@@H](OC)[C@@H]2O)[C@H](O)[C@H]1O]

The associative thickener of structure (B) may have a molecular weight in the range of about 950 g/mole to about 1050 g/mole. When Structure B is the associative thickeners of the present invention, the associative thickener may be present in the personal cleansing composition at a level of about 3.25 wt. % to about 5 wt. %, or about 3.5 wt. % to about 4 wt. %.

The personal cleansing compositions herein may be free of or substantially free of inorganic salt thickeners. For example, the composition may contain less than 1% (e.g., 0% to 0.8%, 0.05% to 0.5%, or even 0.1% to 0.3%) of an inorganic salt thickener such as sodium chloride, potassium chloride, sodium sulfate, ammonium chloride, sodium bromide, combinations of these and the like. In sulfate-free cleansing compositions, inorganic salt can introduce instability to the composition by aiding in the formation of an undesirable coacervate between anionic surfactants and cationic polymers prior to the intended use of the composition. Inorganic salt thickeners can undesirably impact the rheological and performance properties of the present composition as well as the consumer-perceived quality of the product.

### Additional Components

The personal cleansing compositions herein may include additional components suitable for use in personal care compositions. Some non-limiting examples of additional ingredients include, for example, other antimicrobial agents, active agents for providing a hair or scalp benefit, additional surfactants, humectants, emollients, fragrances, stabilizers, colorants, and antioxidants. Such agents and the amounts in which they may be incorporated would be known to those of ordinary skill in the art. For example, an optional additional ingredient may be present at 0.1% to 30% (e.g., 0.5% to 25%, 1% to 20%, 3% to 15%, or 5% to 10%).

The personal care composition may be free of or substantially free of antimicrobial agents or other ingredients that do not meet a particular sustainability standard or naturally derived ingredient standard such as, for example, EWG VERIFIED™, Whole Foods® unacceptable ingredients list, and “risk-free” (green dot) by the Yuka® Application. Some non-limiting examples of antimicrobial agents that may not be suitable for use herein include isothiazolinones (e.g., 5-chloro-2-methyl-4-isothiazolin-3-one and 2-methyl-4-isothiazolin-3-one, commercially available as Kathon™ CG from Dow®), benzyl alcohol, phenoxyethanol, cyclohexylglycerin, parabens, and ethylenediaminetetraacetic acid (EDTA) and salts thereof.

### Dispersed Gel Network

The personal care compositions described herein may include a dispersed gel network phase to provide a cleaning and/or conditioning benefit to the composition in combination with the detersive surfactant. A gel network phase can confer a cleaning benefit to the personal care composition through its hydrophobic nature. Specifically, it is believed, without being limited by theory, that the hydrophobic nature of the dispersed gel network allows the gel network to dissolve hydrophobic soils such as oil into the gel network. Once the soils are dissolved into the gel network, the gel network can be rinsed out of the hair or skin.

Suitable dispersed gel networks can be formed by combining a fatty alcohol and a gel network surfactant in a suitable ratio and heating the dispersion to a temperature above the melting point of the fatty alcohol. During the mixing process, the fatty alcohol melts allowing the gel network surfactant to partition and bring water into the fatty alcohol. Mixing of the gel network surfactant and fatty alcohols also changes the isotropic fatty alcohol drops into liquid crystalline phase drops. When the mixture is subsequently cooled below the melt transition temperature of the fatty alcohols, the liquid crystal phase is converted into a solid crystalline gel network. Additional details of suitable gel networks are described in G. M. Eccleston, “Functions of Mixed Emulsifiers and Emulsifying Waxes in Dermatological Lotions and Creams”, Colloids and Surfaces A: Physiochem. and Eng. Aspects 123-124 (1997) 169-182; and by G. M Eccleston, “The Microstructure of Semisolid Creams”, Pharmacy International, Vol. 7, 63-70 (1986), each of which is incorporated by reference herein.

The presence of a gel network in the pre-mix and in a personal care composition can be confirmed by means known to one of skill in the art. For example, X-ray analysis, optical microscopy, electron microscopy, and differential scanning calorimetry can be used to identify a gel network. A suitable x-ray analysis method is described in U.S. Publication No. 2006/0024256. In some aspects, the scale size of the dispersed gel network in a personal care composition can range from about 10 nm to about 500 nm (e.g., 0.5 μm to 10 μm or 10 μm to about 150 μm).

### Gel Network Fatty Alcohol

The dispersed gel network may include a fatty alcohol (e.g., C10-C40 fatty alcohols) at 0.05% or more by weight of the composition (e.g., 0.05% to about 25%, 0.5% to 20%, or 1% to 8%). The fatty alcohol may be straight or branched chain and can be saturated or unsaturated. As can be appreciated, suitable fatty alcohols can be of natural, vegetable, or synthetic origin. In some aspects, it may be desirable to mix several fatty alcohols to provide a dispersed gel network phase with a melt transition temperature of about 38° C. or greater such as, for example, a mixture of cetyl alcohol and stearyl alcohol at a ratio of between 20:80 and 80:20. Some non-limiting examples of fatty alcohols that may be suitable for use herein include cetyl alcohol, stearyl alcohol, arachidyl alcohol, behenyl alcohol, C₂₁ fatty alcohol (1-heneicosanol), C₂₃ fatty alcohol (1-tricosanol), C₂₄ fatty alcohol (lignoceryl alcohol, 1-tetracosanol), C₂₆ fatty alcohol (1-hexacosanol), C₂₈ fatty alcohol (1-octacosanol), C₃₀ fatty alcohol (1-triacontanol), C₂₀₋₄₀ alcohols (e.g., Performacol® 350 and 425 Alcohols, available from New Phase Technologies), C₃₀₋₅₀ alcohols (e.g., Performacol® 550 Alcohol), C₄₀₋₆₀ alcohols (e.g., Performacol® 700 Alcohol), and mixtures thereof.

### Gel Network Surfactant

The gel network phase may include a gel network surfactant at 0.01% to 15% by weight of the composition (e.g., 0.1% to about 10%, 0.2% to about 5%). The gel network surfactant is combined with the fatty alcohol and liquid carrier to form a gel network pre-mix, which can then be added to the other ingredients of the personal care composition.

The total weight of the gel network surfactant and the fatty alcohols may be 0.5% to about 15% by weight of the personal care composition (e.g., 1% to 10%). In some aspects, the gel network surfactant may be included in the gel network at a desired weight ratio with respect to the fatty alcohols. For example, the ratio of the fatty alcohols to the gel network surfactant may be 1:5 to 100:1 (e.g., 1:1 to 40:1, 2:1 to 20:1, or even 3:1 to 10:1).

The gel network surfactant can be any suitable anionic, zwitterionic, amphoteric, cationic, and nonionic surfactants that is substantially free of sulfates. The detersive surfactant and the gel network surfactant can be the same or different. The gel network surfactant may have a hydrophobic tail group with a chain length of 10 to 40 carbon atoms. The hydrophobic tail group may be alkyl, alkenyl (containing up to 3 double bonds), alkyl aromatic, or branched alkyl. Mixtures of more than one gel network surfactant can also be used. Some non-limiting examples of gel network surfactants are disclosed in U.S. Publication No. 2006/0024256.

### Liquid Carrier for the Gel Network

In some aspects, the dispersed gel network phase may include a suitable liquid carrier at 0.05% to 95% by weight of the personal care composition. The liquid carrier can be water or another suitable solvent. The carrier and the gel network surfactant may be selected to work together to swell the fatty alcohol, which leads to the formation and stability of the gel network phase. A suitable solvent is any that can be used in the place of or in combination with water in the formation of the gel network phase. In some aspects, the liquid carrier can be substantially free of solvents other than water. In some aspects, the liquid carrier for the dispersed gel network phase can be included at a weight ratio of about 1:1 with the fatty alcohol of the dispersed gel network phase.

### Cationic Polymer

A cationic polymer can be used to increase deposition of a dispersed gel network phase or aid in the formation of a coacervate. In formulations including a cationic polymer, the polymer can be included by weight of the personal care composition at about 0.05% to about 3%, about 0.075% to about 2.0%, or at about 0.1% to about 1.0%. Cationic polymers can have cationic charge densities of about 0.9 meq/gm or more, about 1.2 meq/gm or more, and about 1.5 meq/gm or more. However, cationic charge density can also be about 7 meq/gm or less in certain embodiments and about 5 meq/gm or less in certain embodiments. The charge densities can be measured at the pH of intended use of the personal care composition. (e.g., at about pH 3 to about pH 9; or about pH 4 to about pH 8). The average molecular weight of cationic polymers can generally be between about 10,000 and 10 million, between about 50,000 and about 5 million, and between about 100,000 and about 3 million.

Suitable cationic polymers may contain cationic nitrogen-containing moieties such as quaternary ammonium or cationic protonated amino moieties. The cationic protonated amines may be primary, secondary, or tertiary amines (preferably secondary or tertiary), depending upon the particular species and the selected pH of the composition. Anionic counterions can be used in association with the cationic polymers so long as the polymers remain soluble in water, in the composition, and in a coacervate phase of the composition. Examples of suitable counterions include halide counterions (e.g., chloride, fluoride, bromide, iodide).

Non limiting examples of suitable cationic polymers include copolymers of vinyl monomers having cationic protonated amine or quaternary ammonium functionalities with water soluble spacer monomers such as acrylamide, methacrylamide, alkyl and dialkyl acrylamides, alkyl and dialkyl methacrylamides, alkyl acrylate, alkyl methacrylate, vinyl caprolactone or vinyl pyrrolidone.

Suitable cationic protonated amino and quaternary ammonium monomers may include vinyl compounds substituted with dialkylaminoalkyl acrylate, dialkylaminoalkyl methacrylate, monoalkylaminoalkyl acrylate, monoalkylaminoalkyl methacrylate, trialkyl methacryloxyalkyl ammonium salt, trialkyl acryloxyalkyl ammonium salt, diallyl quaternary ammonium salts, and vinyl quaternary ammonium monomers having cyclic cationic nitrogen-containing rings such as pyridinium, imidazolium, and quaternized pyrrolidone, e.g., alkyl vinyl imidazolium, alkyl vinyl pyridinium, alkyl vinyl pyrrolidone salts.

Other suitable cationic polymers may include copolymers of 1-vinyl-2-pyrrolidone and 1-vinyl-3-methylimidazolium salt (e.g., chloride salt) (referred to in the industry by the Cosmetic, Toiletry, and Fragrance Association, “CTFA”, as Polyquaternium-16); copolymers of 1-vinyl-2-pyrrolidone and dimethylaminoethyl methacrylate (referred to in the industry by CTFA as Polyquaternium-11); cationic diallyl quaternary ammonium-containing polymers, including, for example, dimethyldiallylammonium chloride homopolymer, copolymers of acrylamide and dimethyldiallylammonium chloride (referred to in the industry by CTFA as Polyquaternium 6 and Polyquaternium 7, respectively); amphoteric copolymers of acrylic acid including copolymers of acrylic acid and dimethyldiallylammonium chloride (referred to in the industry by CTFA as Polyquaternium 22), terpolymers of acrylic acid with dimethyldiallylammonium chloride and acrylamide (referred to in the industry by CTFA as Polyquaternium 39), and terpolymers of acrylic acid with methacrylamidopropyl trimethylammonium chloride and methylacrylate (referred to in the industry by CTFA as Polyquaternium 47). In certain embodiments, suitable cationic substituted monomers include cationic substituted dialkylaminoalkyl acrylamides, dialkylaminoalkyl methacrylamides, and combinations thereof. Generally, cationic monomers can conform to the formula I:

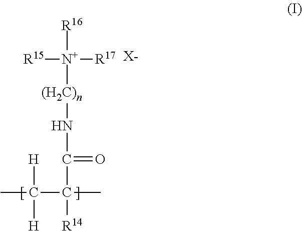

wherein R¹⁴ is hydrogen, methyl or ethyl; each of R¹⁵, R¹⁶ and R¹⁷ are independently hydrogen or a short chain alkyl having from 1 to 8 carbon atoms, or from 1 to 5 carbon atoms, or from 1 to 2 carbon atoms; n is an integer having a value of from 1 to 8, or 1 to 4; and X is a counterion. The nitrogen attached to R¹⁵, R¹⁶ and R¹⁷ can be a protonated amine (primary, secondary or tertiary), but can also be a quaternary ammonium wherein each of R¹⁵, R¹⁶ and R¹⁷ are alkyl groups. The cationic monomer may be polymethyacrylamidopropyl trimonium chloride, available under the trade name Polycare 133, from Rhone-Poulenc, Cranberry, N.J., U.S.A. Copolymers of the cationic monomer may also suitable. The charge density of the total copolymer may be from about 2.0 to about 4.5 meq/gm.

Other cationic polymers may also be suitable including polysaccharide polymers, such as cationic cellulose derivatives and cationic starch derivatives. Suitable cationic polysaccharide polymers include those which conform to Formula J:

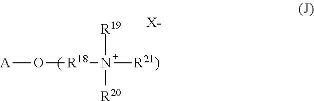

wherein A is an anhydroglucose residual group, such as a starch or cellulose anhydroglucose residual; R¹⁸ is an alkylene oxyalkylene, polyoxyalkylene, or hydroxyalkylene group, or combination thereof; R¹⁹, R²⁰, and R²¹ are independently alkyl, aryl, alkylaryl, arylalkyl, alkoxyalkyl, or alkoxyaryl groups, wherein each group contains up to 18 carbon atoms, and the total number of carbon atoms for each cationic moiety (i.e., the sum of carbon atoms in R¹⁹, R²⁰, and R²¹) is 20 or less; and X is an anionic counterion.

A cationic cellulose polymer may be selected from the salts of hydroxyethyl cellulose reacted with trimethyl ammonium substituted epoxide, referred to in the industry (CTFA) as Polyquaternium 10 and available from Amerchol Corp. (Edison, N.J., USA) in their Polymer LR, JR, and KG series of polymers. Other suitable cationic cellulose polymers may include polymeric quaternary ammonium salts of hydroxyethyl cellulose reacted with lauryl dimethyl ammonium-substituted epoxide referred to in the industry (CTFA) as Polyquaternium 24. These materials are available from Amerchol Corp. under the tradename Polymer LM-200.

Other suitable cationic polymers can include cationic guar gum derivatives, such as guar hydroxypropyltrimonium chloride. Additional examples of suitable cationic guar gum derivatives can include the Jaguar series commercially available from Rhone-Poulenc Incorporated and the N-Hance series commercially available from Aqualon Division of Hercules, Inc. Additional details about cationic guar gum derivatives are disclosed in U.S. Pat. No. 6,930,078 which is incorporated by reference herein.

Quaternary nitrogen-containing cellulose ethers may be suitable. Examples of quaternary nitrogen-containing cellulose ethers are described in U.S. Pat. No. 3,962,418, which is incorporated herein by reference.

A synthetic cationic polymer or derivative thereof can be useful as a cationic polymer. Generally, such synthetic cationic polymers can be included at a concentration of from about 0.025% to about 5%, by weight of a personal care composition.

Suitable synthetic cationic polymers may include polymers which are water-soluble or dispersible, are cationic, and are non-crosslinked. Suitable polymers may be conditioning copolymers comprising: (i) one or more cationic monomer units; and (ii) one or more nonionic monomer units or monomer units bearing a terminal negative charge; wherein said copolymer has a net positive charge. The synthetic cationic polymers may have a cationic charge density of from about 0.5 meq/g to about 10 meg/g, and can have an average molecular weight from about 1,000 to about 5,000,000.

Non-limiting examples of suitable synthetic cationic deposition polymers are described in United States Patent Application Publication No. US 2003/0223951 which is incorporated herein by reference.

Additional suitable cationic polymers may include copolymers of etherified cellulose, guar and starch, some examples of which are described in U.S. Pat. No. 3,958,581, which is incorporated herein by reference. Additional cationic polymers are also described in the CTFA Cosmetic Ingredient Dictionary, 3rd edition, edited by Estrin, Crosley, and Haynes, (The Cosmetic, Toiletry, and Fragrance Association, Inc., Washington, D.C. (1982)), which is incorporated herein by reference.

In formulations including a cationic polymer, the cationic polymers may be soluble in the composition or can be soluble in a complex coacervate phase in the composition formed by interaction of the cationic polymer and a surfactant. Complex coacervates of the cationic polymer can also be formed with other anionic charged materials in the personal care composition.

Techniques for analysis of formation of complex coacervates are known in the art. For example, microscopic analyses of the compositions, at any chosen stage of dilution, can be utilized to identify whether a coacervate phase has formed. Such coacervate phase will be identifiable as an additional emulsified phase in the composition. The use of dyes can aid in distinguishing the coacervate phase from other insoluble phases dispersed in the composition. Additional details about the use of cationic polymers and coacervates are disclosed in U.S. Pat. No. 9,272,164 which is incorporated herein by reference.

### Aqueous Carrier

The composition may optionally include 20-95% of an aqueous carrier such as water and/or a water miscible solvent. The type and amount of aqueous carrier should be selected to provide the composition with the desired rheological properties. The liquid carrier can be water with, e.g., less than 5%, 3%, 1%, 0.5% or even 0% miscible organic solvent. Some nonlimiting examples of organic solvents include lower alkyl alcohols (e.g., ethanol and isopropanol) and polyhydric alcohols (e.g., propylene glycol, hexylene glycol, glycerin, and propane diol).

### Product Forms

The personal care compositions herein may be provided in various product forms such as solutions, suspensions, shampoos, conditioners, lotions, creams, gels, toners, sticks, sprays, aerosols, ointments, cleansing liquid washes, solid bars, pastes, foams, mousses, shaving creams, wipes, strips, patches, hydrogels, film-forming products, facial and skin masks (with and without insoluble sheet), and the like. The composition form may follow from the particular dermatologically acceptable carrier chosen. In some aspects, the personal care compositions described herein may include a dispersed gel network phase that provides a milder, but effective, cleansing benefit to soiled hair in combination with a detersive glycolipid surfactant.

### Method of Making a Personal Care Composition

The personal care composition described herein can be made using conventional methods for making compositions of the type desired (e.g., shampoo, conditioner or body wash). A particularly suitable method of making the compositions herein is described in Example 1 below. In some aspects, the composition may include a gel network to aid in the conditioning of hair or scalp. U.S. Publication No. 2006/269501 discloses methods of making gel networks that may be suitable for use herein.

### Method of Use

The personal care compositions described herein can be used in a conventional manner for cleansing and conditioning of hair or skin. Effective amounts of the composition for use generally range from 1 g to 50 g (e.g., 1 g to 20 g). Generally, a method of treating hair or skin can include applying the personal care composition to the hair or skin. For example, an effective amount of the personal care composition can be applied to the hair or skin, which has been wetted with water, and then the composition can be rinsed off. Application to the hair typically includes working the composition through the hair such that most or all of the hair is contacted with the composition. The personal care composition can be used as a liquid, solid, semi-solid, flake, gel, foam, in a pressurized container with a propellant added, or used in a pump spray form. The viscosity of the product may be selected to accommodate the form desired.

A method for treating the hair or skin can include the steps of: (a) wetting the hair or skin with water; (b) applying an effective amount of the personal care composition to the hair or skin, and (c) rinsing the applied areas of skin or hair with water. These steps can be repeated as many times as desired to achieve the desired cleansing and conditioning benefit.

### Methods

### Method of Making a Personal Care Composition

Tables 1 and 2 show personal cleansing compositions.

Comparative Personal Cleansing Composition 1 can be formed by the following process. DI water is added to a mixing vessel and heated to 75° C.±3° C. while agitating. Sodium Cocoyl Isethionate (SCI) is added to the mixing vessel, and the mixing continues until the SCI has fully dissolved (with no visible particles remaining and batch is clear). After the SCI has fully dissolved, the following materials are added to the mixing vessel: Sodium Benzoate, Sodium Salicylate, Tetrasodium EDTA, Alkyl Amidopropyl Betaine, and Sodium Lauroyl Sarcosinate. The vessel contents are mixed for at least 10 minutes. The batch is then cooled to <35° C. A Polyquaternium-10 slurry is made with water, which is immediately added to the mixing vessel and mixed for 10 minutes. Perfume is then added and mixed in the mixture for at least 2 minutes. Citric Acid is used to titrate the mixture until a pH of 5.5 to 6.0 is reached. DI water is added to bring the final volume to 100%. The mixture is mixed for at least 10 minutes until homogeneity is achieved.

Comparative Personal Cleansing Composition 2 and Inventive Personal Cleansing Composition I can be formed by the following process. Deionized water is added to a mixing vessel while agitating. The following materials are then added to the mixing vessel: Sodium Benzoate, Sodium Salicylate, Tetrasodium EDTA, Alkyl Amidopropyl Betaine, and Rhamnolipids. The vessel contents are mixed for at least 10 minutes. A Polyquaternium-10 slurry is made with water, which is immediately added to the mixing vessel and mixed for 10 minutes. Perfume is then added and mixed in the mixture for at least 2 minutes. Citric acid is used to titrate the mixture until a pH of 6.8 to 7.4 is reached. Deionized water is added to bring the final volume to 100%. The mixture is mixed for at least 10 minutes until homogeneity is achieved.

Comparative Personal Cleansing Composition 3 and Inventive Personal Cleansing Composition 2 can be formed by the following process. Deionized water is added to a mixing vessel while agitating. A Jaguar Excel polymer slurry is made with water, which is immediately added to the mixing vessel and mixed for 10 minutes. The following materials are then added to the mixing vessel: Kathon, Coco Betaine, and Rhamnolipids. The vessel contents are mixed for at least 10 minutes. Perfume is then added and mixed in the mixture for at least 2 minutes. Citric acid is used to titrate the mixture until a pH of 6.8 to 7.4 is reached. Deionized water is added to bring the final volume to 100%. The mixture is mixed for at least 10 minutes until homogeneity is achieved.

Comparative Personal Cleansing Composition 4 and Inventive Personal Cleansing Composition 3 can be formed by the following process. Deionized water is added to a mixing vessel while agitating. A Jaguar Excel polymer slurry is made with water, which is immediately added to the mixing vessel and mixed for 10 minutes. The following materials are then added to the mixing vessel: Kathon, NaLAA, and Rhamnolipids. The vessel contents are mixed for at least 10 minutes. Perfume is then added and mixed in the mixture for at least 2 minutes. Citric acid is used to titrate the mixture until a pH of 6.8 to 7.4 is reached. Deionized water is added to bring the final volume to 100%. The mixture is mixed for at least 10 minutes until homogeneity is achieved.

Comparative Personal Cleansing Composition 5 can be formed by the following process. Deionized water is added to a mixing vessel while agitating. The following materials are then added to the mixing vessel: Alkyl Amidopropyl Betaine, Sodium Lauroyl Taurate, Alpha olefin sulfonate (AOS), Sodium Benzoate, Sodium Salicylate, and Tetrasodium EDTA. The vessel contents are mixed for at least 10 minutes. A Polyquaternium-10 slurry is made with water, which is immediately added to the mixing vessel and mixed for 10 minutes. Perfume is then added and mixed in the mixture for at least 2 minutes. Citric acid is used to titrate the mixture until a pH of 5.0 to 6.0 is reached. Deionized water is added to bring the final volume to 100%. The mixture is mixed for at least 10 minutes until homogeneity is achieved.

Comparative Personal Cleansing Composition 6 and Inventive Personal Cleansing Composition 4 can be formed by the following process. Deionized water is added to a mixing vessel while agitating. The following materials are then added to the mixing vessel: Alkyl Amidopropyl Betaine, Sodium Lauroyl Taurate, Rhamnolipids, Sodium Benzoate, Sodium Salicylate, and Tetrasodium EDTA. The vessel contents are mixed for at least 10 minutes. A Polyquaternium-10 slurry is made with water, which is immediately added to the mixing vessel and mixed for 10 minutes. Perfume is then added and mixed in the mixture for at least 2 minutes. Citric acid is used to titrate the mixture until a pH of 5.0 to 6.0 is reached. Deionized water is added to bring the final volume to 100%. The mixture is mixed for at least 10 minutes until homogeneity is achieved.

Comparative Personal Cleansing Composition 7 and Inventive Personal Cleansing Composition 5 can be formed by the following process. Deionized water is added to a mixing vessel while agitating. The following materials are then added to the mixing vessel: Alkyl Amidopropyl Betaine, Alpha olefin sulfonate (AOS), Rhamnolipids, Sodium Benzoate, Sodium Salicylate, and Tetrasodium EDTA. The vessel contents are mixed for at least 10 minutes. A Polyquaternium-10 slurry is made with water, which is immediately added to the mixing vessel and mixed for 10 minutes. Perfume is then added and mixed in the mixture for at least 2 minutes. Citric acid is used to titrate the mixture until a pH of 5.0 to 6.0 is reached. Deionized water is added to bring the final volume to 100%. The mixture is mixed for at least 10 minutes until homogeneity is achieved.

### Blender Lather Volume

This method can be used to evaluate a foam property that consumers associate with the quality of a shampoo product. This method is described in detail in Klein, Ken, “Evaluating Shampoo Foam,” Cosmetic & Toiletries, Vol. 119, No. 10, p 32-35, 2004.

One (1) gram of the shampoo product is added to 39 grams of DI water at room temperature. This solution is carefully poured into the single-speed Magic Bullet MB1001 blender to minimize the introduction of bubbles and agitated for 10 seconds. The foam is then poured into a 250-mL graduated cylinder, and the volume of the foam is measured and recorded.

### Lather Height and Creaminess

The foaming potential of shampoos does not directly influence the physical behavior of hair fibers. However, shampoo foam can influence a user's perception of hair characteristics. In some instances, it may be helpful to distinguish lather from foam for shampoo evaluation. In particular, foam can generically refer to a mass of gas bubbles in a liquid film matrix, whereas lather more specifically refers to a type of foam formed during shampooing and other processes, wherein the foam consists of small bubbles that are densely packed, thus resisting flow.

This method provides a way to simulate the lather produced by surfactants when used on hair under typical shampooing conditions and quantify certain lather properties. Oil (e.g., sebum) is one of the most common contaminate found on hair that can undesirably affect the lather properties of a shampoo. Thus, this method can be used to evaluate the effect of oil on lather properties.

Lather Height (oil) and Creaminess

100 ml of water (at 100° F.) is placed in a suitable blender (e.g., KitchenAid KSB560CU1 brand food mixer or equivalent), followed by 2 mL of the test composition and 1 mL of extra virgin olive oil. Blend the mixture on “stir” for 30 seconds and record the height of the lather.

To assess creaminess, the lather is poured into a suitable bowl and is visually inspected. Based on the visual inspection, lather creaminess is rated from 0 to 5, where 0 is not at all creamy (bad) and 5 is extremely creamy (good).

### Wet Hair In-Lab Screening (ILS)

This method can be used to determine the cleaning and/or conditioning properties of the personal care compositions herein. In this method, a 20 g hair switch of Caucasian Low Lift Hair Tresses (International Hair Importers and Products, Inc.; Glendale, NY) is wetted with water, treated with a personal care composition and subject to testing in an In-Lab Screening (ILS) sink. The sink has a salon spray head/hose that is held in place but can be directed to run water over a hair tress that hangs from a rod placed over the sink. The tress can be moved in and out of the water as necessary. The water is maintained at a temperature of 38° C. and a flow rate of 5.7 liters per minute. The testing is as follows:
Calibrate ILS sink to 38° C.
Hang the hair tress switch on rod in sink.
Wet hair thoroughly for 30 seconds. Squeegee the hair tress switch once using your index and middle finger (“scissor fingers) from top to bottom to remove excess water. (“Squeegee” means to clamp the tress at the top in between your index and middle finger and stroke down once to remove water.)
Apply 0.1 g/g (product/hair) of the test composition to the front of the switch, from top to bottom.
Milk the hair tress switch for 15 seconds, then flip the bottom of the switch to the top and milk for another 15 seconds. (“Milk” means to grab the top of the tress and stroke it downward while alternating hands to create lather.)
Evaluate Lather Creaminess (look and feel, after 30 seconds of lathering). Scale: 0=No Creaminess−10=Extremely Creamy
Evaluate Lather Combing (with lather still in switch). Using the lowest pressure possible, place comb all the way through the hair (starting at the top) and using a minimal amount of force (comb from top to bottom) comb through hair switch. Scale: 0=hard to comb−10=easy to comb.
Rinse for 30 seconds (while lightly milking the switch).
Squeegee the hair switch tress once with scissor fingers.
Evaluate Slippery Feel. Squeegee once and assess feel. Scale: 0=No Slip−10=Extremely Slippery.
Evaluate Clean Feel Post Rinse. Stroke the hair from top to bottom between the thumb and two fingers with medium pressure. Gauge how clean or dirty the hair feels. Scale: 0=Low (Dirty)-10=High (Clean).
Evaluate Post Rinse Comb. Using the lowest pressure possible, placing comb (wide tooth side) all the way through the hair (front to back), and using a minimal amount of force (top to bottom) comb through hair switch. Scale: 0=Hard-10=Easy.
   Modified Short Time Exposure (mSTE) Assay Study Summary (Ocular Irritation)

Corneal epithelial cytotoxicity can lead to eye irritation following accidental exposure to surfactant-containing formulations. The modified Short Time Exposure (mSTE) method, based on the OECD TG 491, is a cytotoxicity-based assay used for assessment of eye irritation potential of surfactant-dominated formulations. The cytotoxicity-based in vitro assay is performed with confluent monolayer of Statens Seruminstitut Rabbit Cornea (SIRC) cells, cultured in 96-well polycarbonate microplates. Cell viability is quantitatively measured, after extraction from cells, of blue formazan salt produced by the living cells through enzymatic conversion of the vital dye MTT (3-(4,5-Dimethylthiazol-2-yl)-2,5-diphenyltetrazolium bromide) after 5 min exposure. Viability is compared to the solvent control (relative viability) and used to estimate the potential eye hazard of the test item. Modifications to the standard OECD TG assay involve expanding the number of test item dilutions to six to define a complete dose response curve for cell viability, performing curve fitting using a 4-parameter Hill model and identifying the EC₅₀ for the test item (test item dilution that results in 50% cell viability). EC₅₀s for test items are compared to EC₅₀s for benchmark formulations with known and acceptable eye irritation profiles.

### EXAMPLES

TABLE 1
Inventive Examples E1-E3 and Comparative Examples C1-C4

| RAW MATERIALS | | | | | | | |
|---|---|---|---|---|---|---|---|
| (% Active Basis) | C1 | C2 | E1 | C3 | E2 | C4 | E3 |
| Alkyl Amidopropyl | 9.8 | 9.8 | 9.8 | 0.0 | 0.0 | 0.0 | 0.0 |
| Betaine¹ | | | | | | | |
| Sodium Cocoyl | 6.0 | 0.0 | 0.0 | 0.0 | 0.0 | 0.0 | 0.0 |
| Isethionate | | | | | | | |
| Sodium Lauroyl | 2.5 | 0.0 | 0.0 | 0.0 | 0.0 | 0.0 | 0.0 |
| Sarcosinate | | | | | | | |
| Coco Betaine | 0.0 | 0.0 | 0.0 | 6.0 | 6.0 | 0.0 | 0.0 |
| Sodium | 0.0 | 0.0 | 0.0 | 0.0 | 0.0 | 8.0 | 8.0 |
| Lauroamphoacetate | | | | | | | |
| (NaLAA) | | | | | | | |
| Mono-sugar/di-lipid + | 0.0 | 12.0 | 0.0 | 12.0 | 0.0 | 8.0 | 0.0 |
| Di-sugar/di-lipid | | | | | | | |
| Rhamnolipids mixture | | | | | | | |
| I ² | | | | | | | |
| Mono-sugar/mono- | 0.0 | 0.0 | 12.0 | 0.0 | 12.0 | 0.0 | 8.0 |
| lipid + Di-sugar/mono- | | | | | | | |
| lipid Rhamnolipids | | | | | | | |
| mixture I ³ | | | | | | | |
| Polyquaternium-10 | 0.3 | 0.3 | 0.3 | 0.0 | 0.0 | 0.0 | 0.0 |
| Jaguar Excel | 0.0 | 0.0 | 0.0 | 0.3 | 0.3 | 0.3 | 0.3 |
| Sodium Benzoate | 0.8 | 0.8 | 0.8 | 0.0 | 0.0 | 0.0 | 0.0 |
| Sodium Salicylate | 0.5 | 0.5 | 0.5 | 0.0 | 0.0 | 0.0 | 0.0 |
| Tetrasodium EDTA | 0.2 | 0.2 | 0.2 | 0.0 | 0.0 | 0.0 | 0.0 |
| Kathon | 0 | 0 | 0 | 0.03 | 0.03 | 0.03 | 0.03 |
| Perfume | 1.1 | 1.1 | 1.1 | 1.1 | 1.1 | 1.1 | 1.1 |
| Buffer | To pH | To pH | To pH | To pH | To pH | To pH | To pH |
| | 5.5 to 6.0 | 6.8 to 7.4 | 6.8 to 7.4 | 6.8 to 7.4 | 6.8 to 7.4 | 6.8 to 7.4 | 6.8 to 7.4 |
| Water | qs | qs | qs | qs | qs | qs | qs |

TECHNICAL DATA ¹¹

| Product Viscosity (cps) | 4092.0 | <50 | <50 | | <50 | <50 | | <50 | <50 | |
|---|---|---|---|---|---|---|---|---|---|---|
| Blender Lather Volume | 130.0 | 110.0 | 170.0 | (s) | 120.0 | 180.0 | (s) | 125.0 | 170.0 | (s) |
| (no soil) (mL) ^{#} | | | | | | | | | | |
| Lather Blender Height | 8.0 | 3.0 | 8.5 | (s) | 4.0 | 7.0 | (s) | 5.0 | 8.0 | (s) |

| (w/oil) (cm) * | | | | | | | | |
|---|---|---|---|---|---|---|---|---|
| Lather Blender | 5 | 0 | 5 | 1 | 4 | 2 | 5 | |
| Creaminess Rating | | | | | | | | |
| (0-5; 0 = not at all | | | | | | | | |
| creamy, | | | | | | | | |
| 5 = extremely creamy) | | | | | | | | |
| “Slippery Feel Post | 5 | 3 | 3 | 2 | 4 | 2 | 4 | (s) |

| Rinse” wet hair ILS | | | | | | | | |
|---|---|---|---|---|---|---|---|---|
| rating ‡ | | | | | | | | |
| “Conditioned Feel Post | 8 | 4 | 4 | 2 | 4 | (s) | 2 | 3 |

Rinse” wet hair ILS
rating ‡

Mildness mSTE Data ^{§}

| | | | | | | | |
|---|---|---|---|---|---|---|---|
| Log EC50 ± SEM | 0.164 ± 0.010 | 0.271 ± 0.014 | 0.323 ± 0.011 | 0.259 ± 0.020 | 0.343 ±0.012 | 0.375 ± 0.028 | 0.445 ± 0.020 |

¹Mixture of Chainlengths-Amphosol CG (supplier: Stepan)
² Evonik Rheance One rhamnolipids (~50% active), 3.6 wt % Rhamno-mono-lipids/94 wt % Rhamno-di-lipids
³ Hydrolysate of Evonik Rheance One rhamnolipids w/o post-hydrolysis work-up (~100% active), 78.2 wt % Rhamno-mono-lipids/16.5 wt % Rhamno-di-lipids
¹¹ (s) Significant difference is operator noticeable vs Comparative Examples w/Commercial Rhamnolipids
^{#} ≥20 mL difference is considered consumer noticeable
* ≥2 cm difference is considered consumer noticeable
‡ ≥2 pt difference is considered consumer noticeable
^{§} greater Log EC50 value is indicative of lower cytotoxicity (i.e., reduced ocular irritation)

TABLE 2
Inventive Examples E4-E5 and Comparative Examples C5-C7

| RAW MATERIALS | | | | | |
|---|---|---|---|---|---|
| (% Active Basis) | C5 | C6 | E4 | C7 | E5 |
| Alkyl Amidopropyl | 5.00 | 5.00 | 5.00 | 5.00 | 5.00 |
| Betaine¹ | | | | | |
| Sodium Lauroyl Taurate | 4.57 | 4.57 | 4.57 | 0.00 | 0.00 |
| Alpha olefin sulfonate | 3.00 | 0.00 | 0.00 | 3.00 | 3.00 |
| (AOS) | | | | | |
| Mono-sugar/di-lipid + | 0.00 | 3.00 | 0.00 | 4.57 | 0.00 |
| Di-sugar/di-lipid | | | | | |
| Rhamnolipids mixture I ² | | | | | |
| Mono-sugar/mono-lipid + | 0.00 | 0.00 | 3.00 | 0.00 | 4.57 |
| Di-sugar/mono-lipid | | | | | |
| Rhamnolipids mixture I ³ | | | | | |
| Sodium Benzoate | 0.75 | 0.75 | 0.75 | 0.75 | 0.75 |
| Sodium Salicylate | 0.45 | 0.45 | 0.45 | 0.45 | 0.45 |
| Tetrasodium EDTA | 0.16 | 0.16 | 0.16 | 0.16 | 0.16 |
| Polyquatemium-10 | 0.25 | 0.25 | 0.25 | 0.25 | 0.25 |
| Perfume | 1.10 | 1.10 | 1.10 | 1.10 | 1.10 |
| Buffer | To pH 5.0 | To pH 5.0 | To pH 5.0 | To pH 5.0 | To pH 5.0 |
| | to 6.0 | to 6.0 | to 6.0 | to 6.0 | to 6.0 |
| Water | qs | qs | qs | qs | qs |

TECHNICAL DATA ¹¹

| Lather Blender Height | 8.0 | 6.0 | 8.0 | (s) | 3.0 | 7.0 | (s) |
|---|---|---|---|---|---|---|---|
| (w/oil) (cm) * | | | | | | | |
| Lather Blender | 5 | 4.5 | 5 | | 0 | 4 | (s) |
| Creaminess Rating | | | | | | | |
| (0-5; 0 = not at all creamy, | | | | | | | |
| 5 = extremely creamy) | | | | | | | |
| “Slippery Feel at | 9 | 8 | 10 | (s) | 9 | 10 | |
| application” wet hair ILS | | | | | | | |
| rating ‡ | | | | | | | |
| “Slippery Feel While | 7 | 6 | 8 | (s) | 8 | 9 | |
| Rinsing” wet hair ILS | | | | | | | |
| rating ‡ | | | | | | | |
| “Slippery Feel Post | 4 | 5 | 5 | | 5 | 8 | (s) |
| Rinsing” wet hair ILS | | | | | | | |
| rating ‡ | | | | | | | |
| “Ease of Combing” wet | 1 | 3 | 5 | (s) | 2 | 10 | (s) |

hair ILS rating ‡

¹Mixture of Chainlengths - Amphosol CG (supplier: Stepan)
² Evonik Rheance One rhamnolipids (~50% active), 3.6 wt % Rhamno-mono-lipids/94 wt % Rhamno-di-lipids
³ Hydrolysate of Evonik Rheance One rhamnolipids w/o post-hydrolysis work-up (~100% active), 78.2 wt % Rhamno-mono-lipids/16.5 wt % Rhamno-di-lipids
¹¹ (s) Significant difference is operator noticeable vs Comparative Example w/Commercial Rhamnolipids
* ≥2 cm difference is considered consumer noticeable (vs. Comparative Example)
‡ ≥2 point difference is considered consumer noticeable (vs. Comparative Example)

With reference to Tables 1 and 2, Inventive Examples 1-5 which include the Rhamno-Mono-Lipids (Mono-sugar/mono-lipid+Di-sugar/mono-lipid Rhamnolipids Mixture I) exhibit significant lather volume, lather height, and lather creaminess improvements over the Comparative Examples (C2-C4, C6, C7) which include the Commercial Rhamno-Di-Lipid (Mono-sugar/di-lipid+Di-sugar/di-lipid Rhamnolipids Mixture I).

Further, with reference to Table 1 and FIG. 1, Inventive Examples E1-E2 which include the Rhamno-Mono-Lipids (Mono-sugar/mono-lipid+Di-sugar/mono-lipid Rhamnolipids Mixture I) are less cytotoxic to corneal epithelial cells (e.g., less ocular irritation), by exhibiting a greater Log EC50 value, than the Comparative Examples (C2-C3) which include the Commercial Rhamno-Di-Lipid (Mono-sugar/di-lipid+Di-sugar/di-lipid Rhamnolipids Mixture I) and Comparative Example (C1) which does not include any rhamnolipids. Also with reference to Table 1 and FIG. 1, Inventive Example E3 which includes the Rhamno-Mono-Lipids (Mono-sugar/mono-lipid+Di-sugar/mono-lipid Rhamnolipids Mixture I) with Sodium Lauroamphoacetate (NaLAA) are less cytotoxic to corneal epithelial cells (e.g., less ocular irritation) as compared to Comparative Example C4 which includes the Commercial Rhamno-Di-Lipid (Mono-sugar/di-lipid+Di-sugar/di-lipid Rhamnolipids Mixture I) with Sodium Lauroamphoacetate (NaLAA) and even less cytotoxic to corneal cells than Mono-sugar/mono-lipid+Di-sugar/mono-lipid Rhamnolipids Mixture I) alone (E1-E2), Commercial Rhamno-Di-Lipid (Mono-sugar/di-lipid+Di-sugar/di-lipid Rhamnolipids Mixture I) alone (C2-C3), and compositions that do not include any rhamnolipids (C1). As is apparent from Table 1 and FIG. 1, a composition with the Rhamno-Mono-Lipids (Mono-sugar/mono-lipid+Di-sugar/mono-lipid Rhamnolipids Mixture I) provide a statistically significant eye irritation benefit as compared to a similar composition with the Commercial Rhamno-Di-Lipid (Mono-sugar/di-lipid+Di-sugar/di-lipid Rhamnolipids Mixture I) and/or a composition without rhamnolipids. Further, the addition of a co-surfactant, such as NaLAA, to the Rhamno-Mono-Lipids (Mono-sugar/mono-lipid+Di-sugar/mono-lipid Rhamnolipids Mixture I), provides an enhanced eye irritation benefit.

For Inventive Examples 1-3, the rhamnolipids are used as the primary anionic surfactant in the sulfate-free formulations in combination with various cosurfactants (e.g., Alkyl Amidopropyl Betainc. Coco Betainc. NaLAA). At the high Rhamno-Mono-Lipid levels (8.0-12.0%), the Inventive Examples provide conditioning benefits versus the Comparative Examples with the commercially available rhamnolipids (e.g., “Slippery Feel Post Rinse”, “Conditioned Feel Post Rinse”).

For Inventive Examples 4 and 5, the rhamnolipids are used as the secondary and tertiary anionic surfactant in the sulfate-free formulations in combination with various cosurfactants (e.g., AOS, Sodium Lauroyl Taurate). At the low Rhamno-Mono-Lipid levels (3.0-4.6%), the Inventive Examples provide conditioning benefits versus the Comparative Examples with the commercially available rhamnolipids (e.g., “Slippery Feel at application”, “Slippery Feel While Rinsing”, “Slippery Feel Post Rinsing”, “Ease of Combing”). The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

### Examples/Combinations

A. A personal care composition comprising:
a) a mono-rhamno-mono-lipid of structure I:

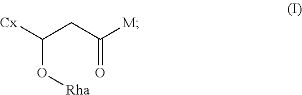

b) a di-rhamno-mono-lipid of structure II:

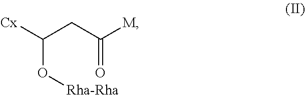

wherein:
Rha is rhamnose,
Cx is a C4-C22 alkyl, aryl, heteroalkyl, heteroaryl, unsaturated alkenyl, or unsaturated heteroalkenyl, and
M is a OH, alkyl, heteroalkyl, aryl, heteroaryl, hetero arylalkyl, arylalkyl, tauryl, O—X+, wherein X+ is a cation, or O—R1, wherein R1 is selected from an alkyl, branched alkyl, and cyclic alkyl, and stereoisomers thereof;
wherein the personal care composition comprises a Log EC50 value of greater than about 0.3.

B. The personal care composition of Paragraph A, wherein the composition has a viscosity in the range of 15 to 50 cps.

C. The personal care composition of any of Paragraphs A through B, wherein the composition has a pH in the range of 6.8 to 7.4.

D. The personal care composition of any of Paragraphs A through C, wherein the co-surfactant is selected from acyl taurate surfactant, an N-alkyl acyl taurate surfactant, an alpha olefin sulfonate surfactant, betaine, sodium lauroamphoacete, and combinations thereof.

E. The personal care composition of Paragraph D, wherein the acyl taurate surfactant comprises lauroyl taurate or a salt thereof.

F. The personal care composition of Paragraph D, wherein the olefin sulfonate is a C14-16 alpha olefin sulfonate.

G. The personal care composition of Paragraph D, wherein the N-alkyl acyl taurate surfactant comprises lauroyl methyl taurate or a salt thereof.

H. The personal care composition of Paragraph D, wherein the betaine surfactant is cocobetaine, cocamidopropyl betaine and mixtures thereof.

I. The personal care composition of any of Paragraphs A through H, wherein the total amount of mono-rhamno-mono-lipid of structure I and di-rhamno-mono-lipid of structure II present is about 0.5 wt. % to about 30 wt. %, preferably about 1 wt. % to about 25 wt. %, more preferably about 2 wt. % to about 20 wt. %, even more preferably about 3 wt. % to about 15 wt. %, and wherein the co-surfactant is present at a level of about 0.5 wt. % to about 30 wt. %, preferably about 1 wt. % to about 25 wt. %, more preferably about 2 wt. % to about 20 wt. %, even more preferably about 3 wt. % to about 15 wt. %.

J. The personal care composition of any of Paragraphs A through I, wherein the total amount of surfactant present in the composition is from about 5 wt. % to about 50 wt. %, preferably about 6 wt. % to about 40 wt. %, more preferably about 7 wt. % to about 30 wt. %, even more preferably about 10 wt. % to about 25 wt. %.

K. The personal care composition of any of Paragraphs A through J, wherein the ratio of the total combined amount of mono-rhamno-mono-lipid of structure I and di-rhamno-mono-lipid of structure II to the total amount of co-surfactant is from about 10:1 to about 1:10, preferably about 5:1 to about 1:5, more preferably about 2:1 to about 1:2.

L. The personal care composition of any of Paragraphs A through K, wherein a ratio of the total amount of mono-rhamno-mono-lipid to the total amount of di-rhamno-mono-lipid is about 10:1 to about 1:10, preferably about 1:5 to about 5:1, more preferably about 1:2 to about 2:1.

M. The personal care composition of any of Paragraphs A through L, wherein the composition has a Lather Height of greater than 2.5 cm according to the Blender Lather Height (oil) method.

N. The personal care composition of any of Paragraphs A through M, wherein the composition further comprises a beta hydroxy fatty acid having a formula III:

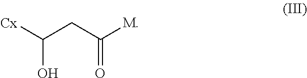

O. The personal care composition of Paragraph N, wherein the beta hydroxy fatty acid is selected from 3-hydroxytetradecanoic acid, 3-hydroxyhexadecanoic acid, 3-hydroxydeceneoic acid, 3-hydroxytetradecenoic acid, 3-hydroxydodecanoic acid, 3-hydroxydodecenoic acid, 3-hydroxyoctanoic acid, 3-hydroxydodec-dienoic acid, and 3-hydroxydecaneoic acid.

P. Use of a personal care composition for reducing ocular irritation during application, the composition comprising:
a) a mono-rhamno-mono-lipid of structure I:

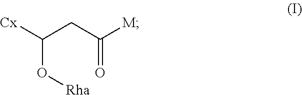

b) a di-rhamno-mono-lipid of structure II:

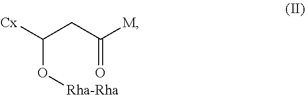

wherein:
Rha is rhamnose,
Cx is a C4-C22 alkyl, aryl, heteroalkyl, heteroaryl, unsaturated alkenyl, or unsaturated heteroalkenyl, and
M is a OH, alkyl, heteroalkyl, aryl, heteroaryl, hetero arylalkyl, arylalkyl, tauryl, O—X+, wherein X+ is a cation, or O—R1, wherein R1 is selected from an alkyl, branched alkyl, and cyclic alkyl, and stereoisomers thereof.

Q. The use of Paragraph P, wherein the personal care composition comprises a Log EC50 value of greater than about 0.3.

R. The use of any of Paragraphs P through Q, wherein the personal care composition comprises a co-surfactant selected from acyl taurate surfactants, olefinsulfonate surfactants, betaine surfactants, lauroamphoacete surfactants, and combinations thereof, preferably acyl taurate surfactant, an N-alkyl acyl taurate surfactant, an alpha olefin sulfonate surfactant, betaine, sodium lauroamphoacete, and combinations thereof.

S. The use of any of Paragraphs P through R, wherein a total amount of mono-rhamno-mono-lipid of structure I and di-rhamno-mono-lipid of structure II present is about 0.5 wt. % to about 30 wt. %, preferably about 1 wt. % to about 25 wt. %, more preferably about 2 wt. % to about 20 wt. %, even more preferably about 3 wt. % to about 15 wt. %, and wherein the co-surfactant is present at a level of about 0.5 wt. % to about 30 wt. %, preferably about 1 wt. % to about 25 wt. %, more preferably about 2 wt. % to about 20 wt. %, even more preferably about 3 wt. % to about 15 wt. %.

T. The use of any of Paragraphs P through S, wherein a total amount of surfactant present in the composition is from about 5 wt. % to about 50 wt. %, preferably about 6 wt. % to about 40 wt. %, more preferably about 7 wt. % to about 30 wt. %, even more preferably about 10 wt. % to about 25 wt. %.

U. The use of any of Paragraphs P through T, wherein a ratio of a total combined amount of mono-rhamno-mono-lipid of structure I and di-rhamno-mono-lipid of structure II to a total amount of co-surfactant is from about 10:1 to about 1:10, preferably about 5:1 to about 1:5, more preferably about 2:1 to about 1:2.

V. The use of any of Paragraphs P through U, wherein a ratio of the total amount of mono-rhamno-mono-lipid to the total amount of di-rhamno-mono-lipid is about 10:1 to about 1:10, preferably about 1:5 to about 5:1, more preferably about 1:2 to about 2:1.

W. The use of any of Paragraphs P through V, wherein the personal care composition has a viscosity of 15 cps to 50 cps.

X. The use of any of Paragraphs P through W, wherein the personal care composition has a pH of 6.8 to 7.4.

Y. The use of any of Paragraphs P through X, wherein the personal care composition further comprises a beta hydroxy fatty acid having a formula III:

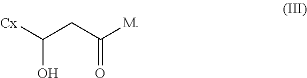

Z. The use of any of Paragraphs P through Y, wherein the personal care composition comprises a Log EC50 value of about 0.3 to about 0.5.

AA. The use of any of Paragraphs P through Z, wherein the personal care composition has a Lather Height of greater than 2.5 cm according to the Blender Lather Height (oil) method.

AB. The use of any of Paragraphs P through AA, wherein the personal care composition is substantially free of sulfated surfactants.

AC. The use of any of Paragraphs P through AB, wherein the personal care composition is substantially free of inorganic salt thickeners.

AD. The use of any of Paragraphs P through AC, wherein the mono-rhamno mono-lipid is selected from 3-(((2R,3R,4R,5R,6S)-3,4,5-trihydroxy-6-methyltetrahydro-2H-pyran-2-yl)oxy)hexadecanoic acid, 3-(((2R,3R,4R,5R,6S)-3,4,5-trihydroxy-6-methyltetrahydro-2H-pyran-2-yl)oxy)octenoic acid, 3-(((2R,3R,4R,5R,6S)-3,4,5-trihydroxy-6-methyltetrahydro-2H-pyran-2-yl)oxy)decenoic acid, 3-(((2R,3R,4R,5R,6S)-3,4,5-trihydroxy-6-methyltetrahydro-2H-pyran-2-yl)oxy)octanoic acid, 3-(((2R,3R,4R,5R,6S)-3,4,5-trihydroxy-6-methyltetrahydro-2H-pyran-2-yl)oxy)tetradecanoic acid, 3-(((2R,3R,4R,5R,6S)-3,4,5-trihydroxy-6-methyltetrahydro-2H-pyran-2-yl)oxy)decanoic acid, 3-(((2R,3R,4R,5R,6S)-3,4,5-trihydroxy-6-methyltetrahydro-2H-pyran-2-yl)oxy)dodecanoic acid, 3-(((2R,3R,4R,5R,6S)-3,4,5-trihydroxy-6-methyltetrahydro-2H-pyran-2-yl)oxy)dodecen-dienoic acid, and stereoisomers thereof, and wherein the di-rhamno mono-lipid is selected from 3-(((2R,3R,4R,5R,6S)-4,5-dihydroxy-6-methyl-3-(((2R,3S,4S,5S,6S)-3,4,5-trihydroxy-6-methyltetrahydro-2H-pyran-2-yl)oxy)tetrahydro-2H-pyran-2-yl)oxy)hexadecanoic acid, 3-(((2R,3R,4R,5R,6S)-4,5-dihydroxy-6-methyl-3-(((2R,3S,4S,5S,6S)-3,4,5-trihydroxy-6-methyltetrahydro-2H-pyran-2-yl)oxy)tetrahydro-2H-pyran-2-yl)oxy)tetradecanoic acid, 3-(((2R,3R,4R,5R,6S)-4,5-dihydroxy-6-methyl-3-(((2R,3S,4S,5S,6S)-3,4,5-trihydroxy-6-methyltetrahydro-2H-pyran-2-yl)oxy)tetrahydro-2H-pyran-2-yl)oxy)decenoic acid, 3-(((2R,3R,4R,5R,6S)-4,5-dihydroxy-6-methyl-3-(((2R,3S,4S,5S,6S)-3,4,5-trihydroxy-6-methyltetrahydro-2H-pyran-2-yl)oxy)tetrahydro-2H-pyran-2-yl)oxy)tetradecenoic acid, 3-(((2R,3R,4R,5R,6S)-4,5-dihydroxy-6-methyl-3-(((2R,3S,4S,5S,6S)-3,4,5-trihydroxy-6-methyltetrahydro-2H-pyran-2-yl)oxy)tetrahydro-2H-pyran-2-yl)oxy)decanoic acid, 3-(((2R,3R,4R,5R,6S)-4,5-dihydroxy-6-methyl-3-(((2R,3S,4S,5S,6S)-3,4,5-trihydroxy-6-methyltetrahydro-2H-pyran-2-yl)oxy)tetrahydro-2H-pyran-2-yl)oxy)octanoic acid, and 3-(((2R,3R,4R,5R,6S)-4,5-dihydroxy-6-methyl-3-(((2R,3S,4S,5S,6S)-3,4,5-trihydroxy-6-methyltetrahydro-2H-pyran-2-yl)oxy)tetrahydro-2H-pyran-2-yl)oxy)dodecanoic acid, and stereoisomers thereof.

AE. A non-therapeutic cosmetic method for washing hair, the method comprising applying to the hair a shampoo composition comprising:
a) a mono-rhamno-mono-lipid of structure I:

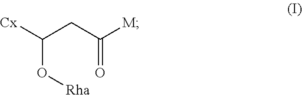

b) a di-rhamno-mono-lipid of structure II:

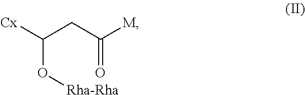

wherein:
Rha is rhamnose,
Cx is a C4-C22 alkyl, aryl, heteroalkyl, heteroaryl, unsaturated alkenyl, or unsaturated heteroalkenyl, and
M is a OH, alkyl, heteroalkyl, aryl, heteroaryl, hetero arylalkyl, arylalkyl, tauryl, O—X+, wherein X+ is a cation, or O—R1, wherein R1 is selected from an alkyl, branched alkyl, and cyclic alkyl, and stereoisomers thereof;
for cleaning hair and scalp while reducing the ocular irritation potential of the composition.

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests, or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A personal care composition comprising:
a) a mono-rhamno-mono-lipid of structure I: 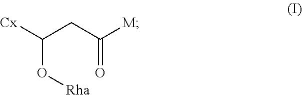
b) a di-rhamno-mono-lipid of structure II: 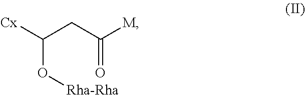
wherein:
Rha is rhamnose,
Cx is a C4-C22 alkyl, aryl, heteroalkyl, heteroaryl, unsaturated alkenyl, or unsaturated heteroalkenyl, and
M is a OH, alkyl, heteroalkyl, aryl, heteroaryl, hetero arylalkyl, arylalkyl, tauryl, O—X+, wherein X+ is a cation, or O—R1, wherein R1 is selected from an alkyl, branched alkyl, and cyclic alkyl, and stereoisomers thereof;
wherein the personal care composition comprises a Log EC50 value of greater than about 0.3.

2. The personal care composition of claim 1, wherein the composition has a viscosity in the range of 15 to 50 cps.

3. The personal care composition of claim 1, wherein the composition has a pH in the range of 6.8 to 7.4.

4. The personal care composition of claim 1, further comprising a co-surfactant chosen from acyl taurate surfactant, an N-alkyl acyl taurate surfactant, an alpha olefin sulfonate surfactant, betaine, sodium lauroamphoacete, an amidopropyl hydroxysultaine, an alkyl glucoside, an amphodiacetate, an ethoxylated alkyl sulfosuccinate, a glutamate, and combinations thereof.

5. The personal care composition of claim 4, wherein the acyl taurate surfactant comprises lauroyl taurate or a salt thereof.

6. The personal care composition of claim 4, wherein the olefin sulfonate is a C14-16 alpha olefin sulfonate.

7. The personal care composition of claim 4, wherein the N-alkyl acyl taurate surfactant comprises lauroyl methyl taurate or a salt thereof.

8. The personal care composition of claim 4, wherein the betaine surfactant is cocobetaine, cocamidopropyl betaine and mixtures thereof.

9. The personal care composition of claim 4, wherein the total amount of mono-rhamno-mono-lipid of structure I and di-rhamno-mono-lipid of structure II present is about 0.5 wt. % to about 30 wt. %, and wherein the co-surfactant is present at a level of about 0.5 wt. % to about 30 wt. %.

10. The personal care composition of claim 4, wherein the total amount of surfactant present in the composition is from about 5 wt. % to about 50 wt. %.

11. The personal care composition of claim 4, wherein the ratio of the total combined amount of mono-rhamno-mono-lipid of structure I and di-rhamno-mono-lipid of structure II to the total amount of co-surfactant is from about 10:1 to about 1:10.

12. The personal care composition of claim 1, wherein a ratio of the total amount of mono-rhamno-mono-lipid to the total amount of di-rhamno-mono-lipid is 10:1 to 1:10.

13. The personal care composition of claim 1, wherein the composition has a Lather Height of greater than 2.5 cm according to the Blender Lather Height (oil) method.

14. The personal care composition of claim 1, wherein the composition further comprises a beta hydroxy fatty acid having a formula III:

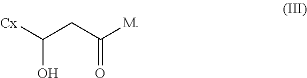

15. The personal care composition of claim 14, wherein the beta hydroxy fatty acid is selected from 3-hydroxytetradecanoic acid, 3-hydroxyhexadecanoic acid, 3-hydroxydeceneoic acid, 3-hydroxytetradecenoic acid, 3-hydroxydodecanoic acid, 3-hydroxydodecenoic acid, 3-hydroxyoctanoic acid, 3-hydroxydodec-dienoic acid, and 3-hydroxydecaneoic acid.

16. The personal care composition of claim 1, wherein at least one of the mono-rhamno-mono-lipid of structure I and di-rhamno-mono-lipid of structure II is present at 25 wt % or more, based on the total weight of rhamnolipids.

17. The personal care composition of claim 1, wherein the total amount of the mono-rhamno, mono-lipid of structure I and the di-rhamno, mono-lipid of structure II is 80 wt % or more, based on the total weight of rhamnolipids.

18. The personal care composition of claim 1, wherein the composition comprises less than 25 wt % of a rhamno di-lipid, based on the total weight of rhamnolipids in the composition.

19. The personal care composition of claim 1, wherein the mono-rhamno mono-lipid is selected from 3-(((2R,3R,4R,5R,6S)-3,4,5-trihydroxy-6-methyltetrahydro-2H-pyran-2-yl)oxy)hexadecanoic acid, 3-(((2R,3R,4R,5R,6S)-3,4,5-trihydroxy-6-methyltetrahydro-2H-pyran-2-yl)oxy)octenoic acid, 3-(((2R,3R,4R,5R,6S)-3,4,5-trihydroxy-6-methyltetrahydro-2H-pyran-2-yl)oxy)decenoic acid, 3-(((2R,3R,4R,5R,6S)-3,4,5-trihydroxy-6-methyltetrahydro-2H-pyran-2-yl)oxy)octanoic acid, 3-(((2R,3R,4R,5R,6S)-3,4,5-trihydroxy-6-methyltetrahydro-2H-pyran-2-yl)oxy)tetradecanoic acid, 3-(((2R,3R,4R,5R,6S)-3,4,5-trihydroxy-6-methyltetrahydro-2H-pyran-2-yl)oxy)decanoic acid, 3-(((2R,3R,4R,5R,6S)-3,4,5-trihydroxy-6-methyltetrahydro-2H-pyran-2-yl)oxy)dodecanoic acid, 3-(((2R,3R,4R,5R,6S)-3,4,5-trihydroxy-6-methyltetrahydro-2H-pyran-2-yl)oxy)dodecen-dienoic acid, and stereoisomers thereof and wherein the di-rhamno mono-lipid is selected from 3-(((2R,3R,4R,5R,6S)-4,5-dihydroxy-6-methyl-3-(((2R,3S,4S,5S,6S)-3,4,5-trihydroxy-6-methyltetrahydro-2H-pyran-2-yl)oxy)tetrahydro-2H-pyran-2-yl)oxy)hexadecanoic acid, 3-(((2R,3R,4R,5R,6S)-4,5-dihydroxy-6-methyl-3-(((2R,3S,4S,5S,6S)-3,4,5-trihydroxy-6-methyltetrahydro-2H-pyran-2-yl)oxy)tetrahydro-2H-pyran-2-yl)oxy)tetradecanoic acid, 3-(((2R,3R,4R,5R,6S)-4,5-dihydroxy-6-methyl-3-(((2R,3S,4S,5S,6S)-3,4,5-trihydroxy-6-methyltetrahydro-2H-pyran-2-yl)oxy)tetrahydro-2H-pyran-2-yl)oxy)decenoic acid, 3-(((2R,3R,4R,5R,6S)-4,5-dihydroxy-6-methyl-3-(((2R,3S,4S,5S,6S)-3,4,5-trihydroxy-6-methyltetrahydro-2H-pyran-2-yl)oxy)tetrahydro-2H-pyran-2-yl)oxy)tetradecenoic acid, 3-(((2R,3R,4R,5R,6S)-4,5-dihydroxy-6-methyl-3-(((2R,3S,4S,5S,6S)-3,4,5-trihydroxy-6-methyltetrahydro-2H-pyran-2-yl)oxy)tetrahydro-2H-pyran-2-yl)oxy)decanoic acid, 3-(((2R,3R,4R,5R,6S)-4,5-dihydroxy-6-methyl-3-(((2R,3S,4S,5S,6S)-3,4,5-trihydroxy-6-methyltetrahydro-2H-pyran-2-yl)oxy)tetrahydro-2H-pyran-2-yl)oxy)octanoic acid, and 3-(((2R,3R,4R,5R,6S)-4,5-dihydroxy-6-methyl-3-(((2R,3S,4S,5S,6S)-3,4,5-trihydroxy-6-methyltetrahydro-2H-pyran-2-yl)oxy)tetrahydro-2H-pyran-2-yl)oxy)dodecanoic acid, and stereoisomers thereof.

20. A method for cleaning hair and scalp while reducing the eye irritation potential comprising:
(a) providing the personal care composition of claim 1;
(b) dispensing the personal care composition into a palm or cleaning implement;
(c) applying the personal care composition onto wet hair and massaging the personal care composition across the hair and scalp; wherein the shampoo composition is diluted forming a lyotropic liquid crystal coacervate that is deposited onto the hair; and
(d) rinsing the personal care composition from the hair and scalp.
